(19) United States
(12) Patent Application Publication
(10) Pub. No.: US 2009/0018118 A1
(43) Pub. Date: Jan. 15, 2009

(54) HETEROCYCLIC COMPOUNDS

(76) Inventors: Uros Urleb, Ljubljana (SI); Darko Kocjan, Ljubljana (SI); Tina Korosec, Ljubljana (SI); Damjana Rozman, Ljubljana (SI); Jure Acimovic, Ptuji (SI); Alenka Tomazic, Ljubljana (SI); Breda Rode, Kamnik (SI)

Correspondence Address: MANELLI DENISON & SELTER, 2000 M STREET NW SUITE 700, WASHINGTON, DC, 20036-3307, US

(21) Appl. No.: 12/159,409

(22) PCT Filed: Dec. 27, 2006
(86) PCT No.: PCT/EP2006/012541

§ 371 Date: Sep. 18, 2008

(30) Foreign Application Priority Data
Dec. 29, 2005 (EP) 05028643.4

(51) Int. Cl. A61K 31/551; C07D 401/14; A61K 31/496; C07D 401/06; A61K 31/5377; C07D 401/04

(52) U.S. Cl. 514/218; 544/360; 514/253.01; 544/121; 514/235.8; 540/575

(57) ABSTRACT

The invention is related to novel substituted diazaheterocycles useful as effective antihypercholesterolemic agents, methods of their preparation, and pharmaceutical compositions containing them.

The invention is related to novel diazaheterocyclic compounds useful as inhibitors of cholesterol biosynthesis and also useful in providing medicaments for the treatment of hypercholesterolemia, hyperlipidemia and related medical pathophysiological conditions in humans. Due to the fact that high blood cholesterol level is a recognized risk factor in the onset of atherosclerosis and because there is a substantial part of nonresponders to existing drugs, there is a constant need for new effective antihypercholesterolemic and antihyperlipidemic agents which would provide a more target-oriented action in the therapy and having fewer side effects in comparison to the active substances known in the prior art.

Several inhibitors of cholesterol biosynthesis are known at the level of inhibition of 3-hydroxy-3-methylglutaryl-coenzyme A reductase (HMG-CoA reductase), as disclosed, for example, in U.S. Pat. No. 4,231,938 (lovastatin), U.S. Pat. No. 4,444,784 (simvastatin), U.S. Pat. No. 4,346,227 (pravastatin sodium) or U.S. Pat. No. 5,273,995 atorvastatin) which are already used in the therapy and are recognized commercial preparations Mevacor®, Simvacor®, Lipitor®. These HMG-CoA reductase inhibitors, also known by the common name statins, significantly lower blood cholesterol levels.

The present invention is directed to provide molecules that will effectively shut down cholesterol synthesis in hepatic tissue in mammals but allow for the build up of the isoprenes needed for the biosynthesis of polyisoprenes other than sterols. Therefore these novel compounds will exhibit less side effects than the action of known statins which inhibit HMG-CoA reductase in an early stage of cholesterol biosynthesis pathway. It is known that lack of sterol intermediates can cause serious side effects. The main goal is to provide drug candidates which inhibit cholesterol biosynthesis in a later step than statins.

Novel compounds of this invention are showing an improved sterol profile, this is a strong effect on cholesterol biosynthesis leading to very low or almost no de novo cholesterol level and also no significant accumulation of post-lanosterol intermediates. Dual- or multi-action inhibitors of cholesterol biosynthesis are preferred. It is well known that lack of sterol intermediates as well as significant accumulation of lanosterol and other post-lanosterol (desmosterol) intermediates can cause serious side effects. Examples are cholestenone^{Δ4-5} and triparanol (MER 29), inhibitors of cholesterol biosynthesis which block the final step in the pathway, namely, the conversion of desmosterol to cholesterol. Application of these inhibitors results in reduction of cholesterol biosynthesis and accumulation of desmosterol in the tissues, such as the vascular walls (atherogenic). This is prohibitive for their clinical use. Triparanol was marketed in US in 1959 as a cholesterol lowering agent. The drug was withdrawn in 1962 because it cause cataracts. (Coleman, V. J Roy Soc Health 1995, 115 (4), 270-270 and Cenedella, R. J. Survey of ophthalmology 1996, 40 (4), 320-337). Clinically used systemic antifuingal agents, such as fluconazole, inhibitor of lanosterol 14α-demethylase, may produce endocrine-related side effect, such as depletion of testosterone and glucocorticoids, resulting in gynecomastia and adrenal insufficiency, respectively. (Georgopapadakou, N. H.; Walsh, T. J. Antimicrobial Agents and Chemotherapy 1996, 40 (2), 279-291).

The problem has been solved by the present invention which relates to novel compounds, to the processes for their preparation, to the pharmaceutical compositions containing them and the use of the compounds in accordance with the invention for the treatment of hypercholesterolemia and hyperlipidemia.

Novel compounds of this invention are compounds with general formula I

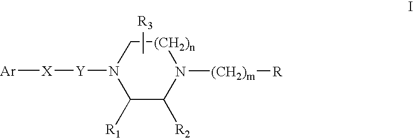

wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none.
R₁ and R₂ are both H or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl;
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₆alkylsulphonyl, carboxyl and C₁₋₆ alkoxycarbonyl group;

The term C₁₋₆ alkyl group includes methyl, ethyl, n-propyl, i-propyl, n-butyl, t-butyl, i-butyl, pentyl and hexyl groups.

The term C₁₋₆ alkoxy group includes methoxy, ethoxy, propoxy, i-propoxy, n-butoxy, t-butoxy, i-butoxy, pentanoxy and hexanoxy groups.

The term aryl group includes substituted or unsubstituted phenyl, naphthyl, anthracenyl groups.

The term heteroaryl group includes furyl, thienyl, pyrrolyl, thiazolyl, pyridyl, pyrimidinyl, pyrazinyl, and oxazolyl which may be fused with a substituted or unsubstituted benzene ring, or a phthalimido group.

The term C₄-C₅ heterocycloalkyl group includes tetrahydrofuranyl, tetrahydropyranyl, piperidinyl, piperazinyl, morpholino and pyrrolidinyl groups.

The term cycloalkyl group includes cyclopropyl, cyclobutyl, cyclopentyl, cyclohexyl, cycloheptyl, cyclooctyl and substituted cycloalkyl groups.

In a preferred embodiment, substituent Ar is a substituted or unsubstituted pyridyl moiety and R is a substituted or unsubstituted phenyl moiety.

The compounds of formula I form salts with all pharmaceutically acceptable acids and these salts are also part of the invention. Such salts are the salts with mineral acids such as, for example, hydrochloric acid, hydrobromic acid, phosphoric acids; or with organic acids such as, for example, methanesulfonic acid, citric acid, oxalic acid, maleic acid, benzenesulfonic acid and others.

New compounds of the present invention may contain one or more asymmetric atoms and can, therefore, exist in racemic form or in the form of optically active enantiomers or diastereomers.

In another aspect the present invention provides a compound selected from the group consisting of
2-(4-Phenethylpiperazin-1-yl)-1-(pyridine-3-yl)ethanol (internal code LK-980B) and a trihydrobromide salt thereof (internal code LK-980)

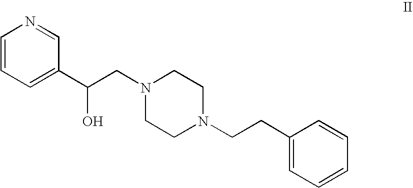

1-Phenethyl-4-(2-(pyridine-3-yl)ethyl)piperazine (internal code LK-9100B) and a trihydrobromide salt thereof (internal code LK-9100)

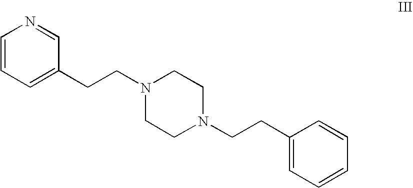

1-Phenethyl-4-(2-(pyridine-4-yl)ethyl)piperazine (internal code LK-9118B) and a trihydrobromide salt thereof (internal code LK-9118)

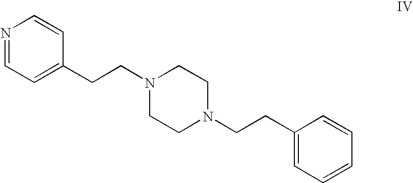

1-Phenethyl-4-(pyridine-3-ylmethyl)piperazine (internal code LK-9108B) and a trihydrobromide salt thereof (internal code LK-9108)

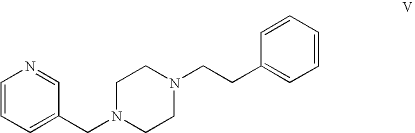

4-(5-((4-Phenethylpiperazin-1-yl)methyl)pyridine-2-yl)morpholine (internal code LK-9109B) and a trihydrobromide salt thereof (internal code LK-9109)

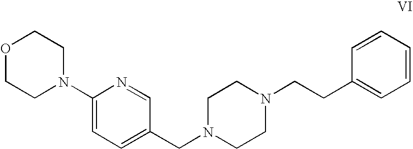

1-Phenethyl-4-((6-(trifluoromethyl)pyridine-3-yl)methyl)piperazine (internal code LK-9107B) and a trihydrobromide salt thereof (internal code LK-9107)

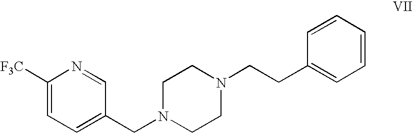

1-(3-Chloro-5-(trifluoromethyl)pyridine-2-yl)-4-phenethyl-1,4-diazepane (internal code LK-9115B) and a trihydrobromide salt thereof (internal code LK-9115)

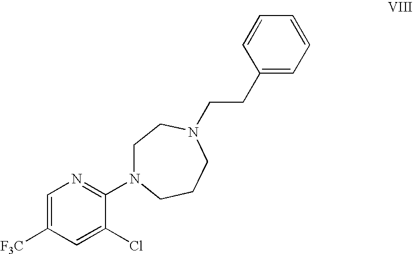

1-(Naphthalen-2-yl)-2-(4-phenethylpiperazine-1-yl)ethanol (internal code LK-9111 B) and a dihydrobromide salt thereof (internal code LK-9111)

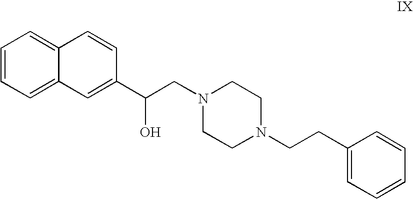

2-(4-Phenethylpiperazin-1-yl)-1-(pyridine-4-yl)ethanol (internal code LK-9110B) and a trihydrobromide salt thereof (internal code LK-9110)

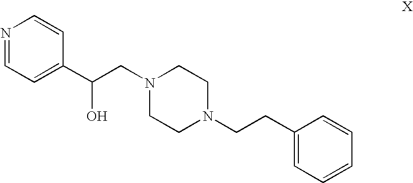

1-Phenethyl-4-(3-(pyridine-3-yl)propyl)piperazine trihydrobromide (LK-9140)
1-Phenethyl-4-(3-(pyridine-3-yl)propyl)piperazine (LK-9140B)

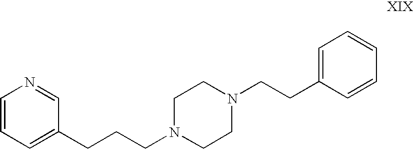

2-(4-(4-Fluorophenyl)piperazine-1-yl)-1-(pyridine-3-yl)ethanol trihydrobromide (LK-9144)
2-(4-(4-Fluorophenyl)piperazine-1-yl)-1-(pyridine-3-yl)ethanol (LK-9144B)

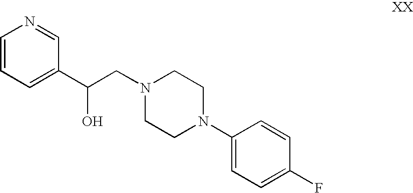

2-(4-(4-Fluorobenzyl)piperazine-1-yl)-1-(pyridine-3-yl)ethanol trihydrobromide (LK-9148)
2-(4-(4-Fluorobenzyl)piperazine-1-yl)-1-(pyridine-3-yl)ethanol (LK-9148B)

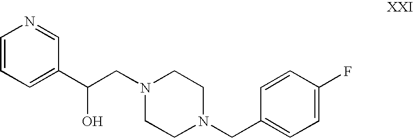

2-(4-Phenethylpiperazine-1-yl)-1-(6-methylpyridine-3-yl)ethanol trihydrobromide (LK-9139)
2-(4-Phenethylpiperazine-1-yl)-1-(6-methylpyridine-3-yl)ethanol (LK-9139B)

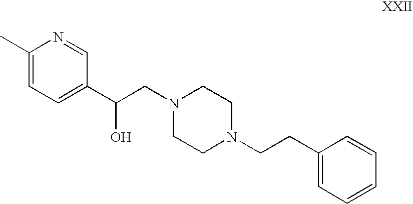

1-(3,4-Difluorophenethyl)-4-(2-(pyridine-4-yl)ethyl)piperazine trihydrobromide (LK-9131)
1-(3,4-Difluorophenethyl)-4-(2-(pyridine-4-yl)ethyl)piperazine (LK-9131B)

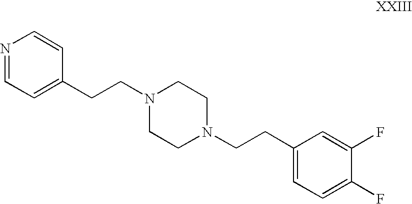

1-(3,4-Dichlorophenethyl)-4-(2-(pyridine-2-yl)ethyl)piperazine trihydrobromide (LK-9137)
1-(3,4-Dichlorophenethyl)-4-(2-(pyridine-2-yl)ethyl)piperazine (LK-9137)

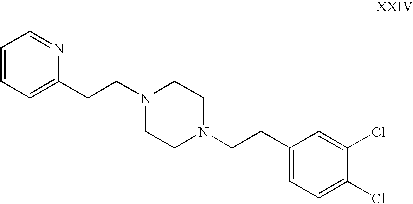

1-(3,4-Difluorophenethyl)-4-(2-(pyridine-2-yl)ethyl)piperazine trihydrobromide (LK-9138)
1-(3,4-Difluorophenethyl)-4-(2-(pyridine-2-yl)ethyl)piperazine (LK-9138B)

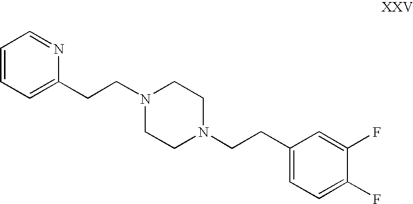

1-(3,4-Dichlorophenethyl)-4-(2-(pyridine-4-yl)ethyl)piperazine trihydrobromide (LK-9135)
1-(3,4-Dichlorophenethyl)-4-(2-(pyridine-4-yl)ethyl)piperazine (LK-9135B)

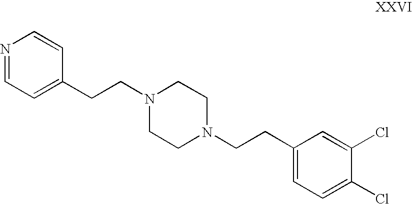

1-((6-Methoxypyridin-3-yl)methyl)-4-phenethyl-piperazine and a trihydrobromide salt thereof (internal code LK 9106)

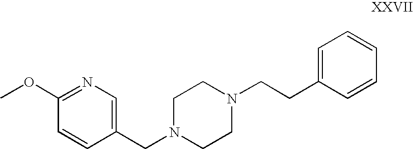

1-Phenethyl-4-(2-(piperidin-3-yl)ethyl)piperazine

The compounds of the invention may be prepared by general methods of synthesis as disclosed below:

### Method a:

Alkylating cyclic secondary amines of formula XI

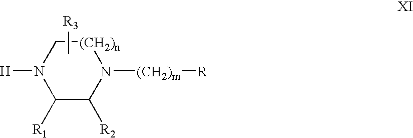

wherein n, m, R₁, R₂, R₃ and R are defined above,
with aryloxirane, heteroaryloxirane of formula XII

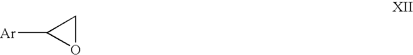

wherein Ar is as defined above,
to the desired arylethanol amines or heteroarylethanol amines of formula I and optionally converting them into the physiologically acceptable acid addition salts thereof.

Aryloxirane and heteroaryloxirane of formula XII in the process of alkylating secondary amines of formula XI are prepared in situ by transformation at bromo-acetylheteroaryl hydrobromide or bromo-acetylaryl with complex metal hydrides, such as sodium borohydride in an inert solvent such as lower aliphatic alkanol, for example, ethanol at a temperature about room temperature.

Bromo-acetylheteroaryl hydrobromide and bromo-acetylaryl are prepared by bromination with bromine and hydrobromic acid of the original acetylaryl or heteroacetylaryl wherein acetylaryl and acetylheteroaryl can be further substituted by up to four substituents as defined above. Substituted or nonsubstituted acetylaryl and acetylheteroaryl are known and commercially available chemicals.

The alkylation step of cyclic secondary amines of formula XI with aryloxirane or heteroaryloxirane of formula XII is carried out at a temperature of about room temperature to reflux temperature of the reaction mixture, in an inert solvent such as lower aliphatic alkanol, for example, ethanol. The crude arylethanol amines or heteroarylethanol amines of formula I are isolated and purified and then, if desired, they are converted into the physiologically acceptable acid addition salts thereof.

### Method b:

Coupling cyclic secondary amines of formula XI

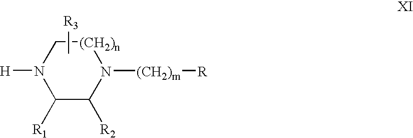

wherein n, m, R₁, R₂, R₃ and R are as defined above,

with carboxylic acid derivatives of formula XIII

Ar—X—COOH XIII

wherein Ar and X are as defined above,
to new intermediate compounds of formula XIV

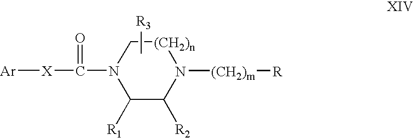

wherein Ar, X, n, m, R₁, R₂, R₃ and R are as defined above,
and reducing them to the Ar-alkyl compounds of formula I and optionally converting them into the physiologically acceptable acid addition salts thereof.

Compounds of formula XIV are prepared by coupling cyclic secondary amines of formula XI with corresponding carboxylic acid derivatives of formula XIII using a coupling reagent, such as 1-ethyl-3-(3-dimethylaminopropyl)carbodiimide (EDC) and 1-hydroxy-1H-benzotriazole (HOBT) activation. Reaction is carried out at a temperature of about room temperature of the reaction mixture, in an inert solvent such as DMF.

The carbonyl group in the novel intermediary compounds XIV may be further reduced. The reaction is carried out with conventional reducing agents. Especially suitable is borane-dimethyl sulfide complex in an inert solvent, such as THF, diethylether and similar. The desired compounds of formula I are isolated and purified and then, if desired, they are converted into the physiologically acceptable acid addition salts thereof.

### Method c:

Alkylating cyclic secondary amines of formula XI

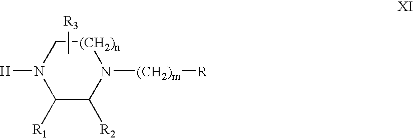

wherein n, m, R₁, R₂, R₃ and R are as defined above,
with formyl derivatives of formula XV

Ar—X—CHO XV

wherein Ar and X are as defined above,
to compounds of formula I
wherein Y is CH₂ and Ar, X, n, m, R₁, R₂, R₃ and R are as defined above,
and optionally converting them into the physiologically acceptable acid addition salts thereof.

Compounds of formula XVI are prepared with reductive alkylation of the cyclic secondary amine of formula XI utilized corresponding formyl derivatives of formula XV and selective hydride reducing agent, such as sodium triacetoxyborohydride. The reaction is carried out at a temperature of about room temperature of the reaction mixture, in an inert solvent such as 1,2-dichloroethane. The crude compounds of formula I are isolated and purified and then, if desired, they are converted into the physiologically acceptable acid addition salts thereof.

### Method d:

Coupling cyclic secondary amines of formula XVI

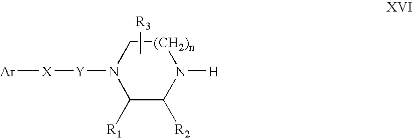

wherein Ar, X, Y, n, R₁, R₂ and R₃ are as defined above,
with carboxylic acid derivatives of formula XVII

COOH—(CH₂)ₘ₋₁—R XVII

wherein R and m are as defined above,
to new intermediate compounds of formula XVIII

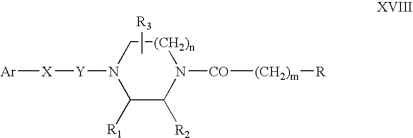

wherein Ar, X, Y, n, m, R₁, R₂, R₃ and R are as defined above,
and reducing them to the desired compounds of formula I and, if desired, converting them into the physiologically acceptable acid addition salts thereof.

Compounds of formula XVIII are prepared by coupling cyclic secondary amines of formula XVI with corresponding carboxylic acid derivatives of formula XVII using a coupling reagent, such as EDC and HOBT activation. Reaction is carried out at a temperature of about room temperature of the reaction mixture, in an inert solvent such as DMF.

A carbonyl group in the novel intermediary compounds of formula XVIII may be further reduced. The reaction is carried out with conventional reducing agents. Especially suitable is borane-dimethyl sulfide complex in an inert solvent, such as THF, diethylether and similar. The desired compounds of formula I are isolated and purified by column chromatography and then, if desired, they are converted into the physiologically acceptable acid addition salts thereof.

The processes for preparation of the novel compounds of formula I in accordance with the variants (a), (b), (c) and (d) are shown in schemes I, II, III, IV.

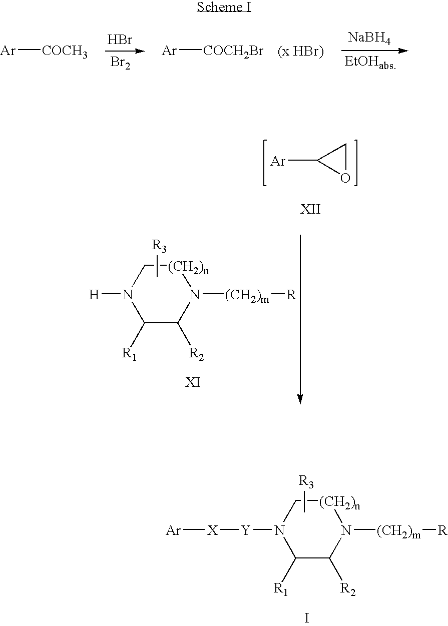

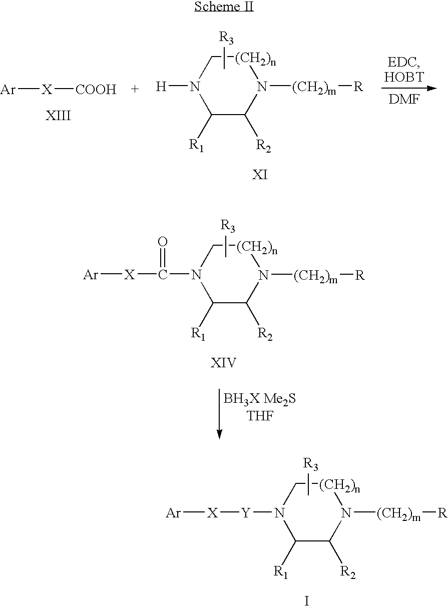

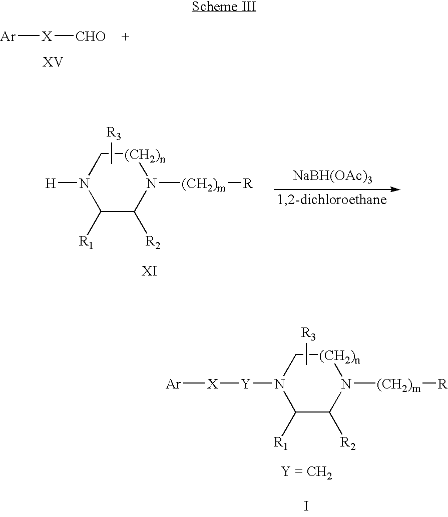

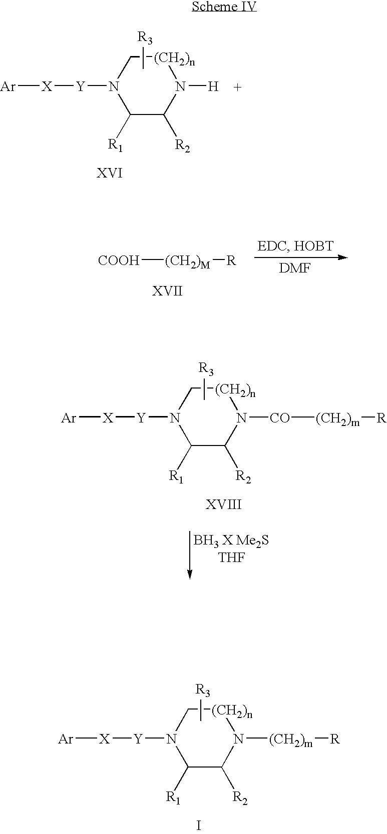

In accordance with the aims of the invention, the effect of the novel compounds on inhibition of cholesterol biosynthesis is assessed. An ex vivo method of metabolic labeling of immortal human hepatocytes is employed. The radioactively labeled early precursor of cholesterol [3H]acetate is added to cells with or without addition of an active new compound.

Preferred sterol profile for the novel substances is an inhibition of cholesterol biosynthesis leading to lowered cholesterol level and no accumulation of lanosterol and other post-lanosterol intermediates. As a preferred compound, LK-9107 meets these criteria in considerable extent.

Application of the novel compounds of formula I of this invention markedly decreases of pathologically increased blood cholesterol levels in patients. The dosage and frequency of application depend on the characteristics of an individual drug, its bioavailability and pharmacokinetic characteristics, and patient's condition.

With novel substituted diazaheterocycles of the present invention a more selective action with fewer side effects is provided due to the inhibition of cholesterol biosynthesis in late steps of this biosynthesis pathway. Consequently, these substances are particularly useful for the treatment of hypercholesterolemia and hyperlipidemia. These effects of the novel diazaheterocycles were truly unexpected as insofar in medical practice and therapy there is a lack of substances that would lower cholesterol level by targeting enzymes in late steps of cholesterol biosynthesis.

Pharmaceutical compositions contain the active substances of the present invention together with the physiologically compatible organic or inorganic support, such as water, lactose, starch and its derivatives, magnesium stearate, talc, plant oils and similar excipients.

Pharmaceutical compositions are preferably administered orally, such as in the form of tablets, capsules, pills, powders, granulates, solutions, syrups, suspensions, elixirs and similar. Administration can be also carried out parenterally, for example, in the form of sterile solutions, suspensions or emulsions. Pharmaceutical preparations can be sterilized and/or can include ingredients, such as preservatives, stabilizers, emulsifiers, buffering substances and other additives.

In another aspect of the invention the pharmaceutical composition contains another pharmaceutically active agent.

The invention is further described by reference to the following examples. These examples are provided for illustration purposes only and are not intended to be limiting the present invention in any way.

### EXAMPLE 1

### 2-(4-Phenethylpiperazine-1-yl)-1-(pyridine3-yl)-1-ethanol trihydrobromide (LK-980)

Preparation of 2-Bromo-1-(3-pyridinyl)-1-ethanone hydrobromide as starting material is exemplary published in e.g. WO 2004/007456.
Preparation of 2-(4-Phenethyl-1-piperazinyl)-1-(3-pyridinyl)-1-ethanol

To a solution of 2-Bromo-1-(3-pyridinyl)-1-ethanone hydrobromide (4.0 g, 14.4 mmol) in anhydrous ethanol (80 ml) is added NaBH₄ (2.0 g, 52.8 mmol). The reaction mixture is stirred at RT for 2 h. The mixture is filtered and 1-(2-Phenylethyl)piperazine (4.9 ml, 26.0 mmol) is added to the filtrate. The solution is heated to reflux and refluxed for 5 h. Excessive ethanol is removed by distillation. The resulting pale yellow solid is dissolved in chloroform (80 ml), the insoluble parts are filtered off and the filtrate is concentrated by distillation under reduced pressure. The product is purified by chromatography on silica (MeOH/EtOAc, 10:2 and MeOH/EtOAc, 1:1) to give a pale yellow solid; yield: 2.0 g, 44% (97% pure by area % HPLC analysis); chemical formula: C₁₉H₂₅N₃O; molecular weight: 311.42.

¹H NMR (300 MHz, CDCl₃) δ 2.48-2.90 (14H, m), 4.80 (1H, dd, J=10.3 Hz, J=3.7 Hz), 7.17-7.33 (6H, m), 7.73 (1H, td, J=7.9 Hz, J=1.7 Hz), 8.53 (1H, dd, J=4.9 Hz, J=1.7 Hz), 8.60 (1H, d, J=2.1 Hz); FT-IR (NaCl) 3027, 2816, 1578, 1427, 1355, 1163, 1094, 1006, 942, 851, 753, 701, 510 cm⁻¹; FAB MS m/z 312 [MH⁺]; HRMS m/z calcd for C₁₉H₂₆N₃O [MH⁺] 312.2076, found 312.2083.
Preparation of 2-(4-Phenethylpiperazine-1-yl)-1-(pyridine-3-yl)-1-ethanol trihydrobromide

A solution of 2-(4-Phenethyl-1-piperazinyl)-1-(3-pyridinyl)-1-ethanol (1.7 g, 5.46 mmol) in acetone (approximate 10 ml) is cooled in an ice bath. 7.5 ml (1.7 g HBr, 21.6 mmol) solution of HBr in ethanol is added dropwise. When precipitation starts, approximately 10 ml of diethyl ether is added. The reaction mixture is stirred in ice for 2 h. The white precipitate is filtered and successively washed with diethyl ether; yield: 1.26 g, 42% (98.5% pure by area % HPLC analysis); mp 178-181° C.; chemical formula: C₁₉H₂₈Br₃N₃O; molecular weight: 554.16.

¹H NMR (300 MHz, DMSO-d₆) δ 3.00-4.14 (14H, m), 5.50 (1H, d, J=8.7 Hz), 7.25-7.38 (5H, m), 8.06-8.15 (1H, m), 8.65 (1H, m), 8.94 (1H, d, J=5.5 Hz), 9.05 (1H, s); FT-IR (NaCl) 3255, 2978, 2436, 1635, 1560, 1438, 1261, 1071, 950, 769, 634 cm⁻¹; FAB MS m/z 312 [MH⁺].

### EXAMPLE 2

### 1-Phenethyl-4-(2-(pyridine-3-yl)ethyl)piperazine trihydrobromide (LK-9100)

Preparation of 1-(4-Phenethylpiperazin-1-yl)-2-(pyridine-3-yl)ethanone (LK-9101B)

To a solution of 1-(2-Phenylethyl)piperazine (2.2 ml, 11.5 mmol) and 3-pyridylacetic acid hydrochloride (2.0 g, 11.5 mmol) in DMF (approximately 15 ml) is added HOBT (1.6 g, 11.5 mmol). The pH of the solution is adjusted to 8 by adding N-methylmorpholine. Coupling reagent EDC (2.3 g, 11.9 mmol) is added. After the reaction mixture is stirred at RT over night, the solvent is evaporated under reduced pressure and the residue is dissolved in EtOAc (20 ml). The organic layer is washed with aqueous saturated NaHCO₃ (20 ml) and NaCl (20 ml) solution and dried (Na₂SO₄). The solvent is evaporated under reduced pressure to give a product which is chromatographed on silica (MeOH/EtOAc, 2:10) to give a brownish solid; yield: 3.56 g, 100% (98% pure by area % HPLC analysis); mp 91-93° C.; chemical formula: C₁₉H₂₃N₃O; molecular weight: 309.41.

¹H NMR (300 MHz, DMSO-d₆) δ 2.37-2.43 (4H, m), 2.50-2.54 (4H, m), 2.71-2.76 (2H, m), 3.40-3.54 (2H, m), 3.76 (2H, s), 7.18-7.35 (6H, m), 7.61 (1H, td, J=7.8 Hz, J=1.7), 8.42-8.44 (2H, m); FT-IR (NaCl) 3409, 2776, 1652, 1579, 1424, 1311, 1237, 1134, 1237, 1134, 998, 767, 697 cm⁻¹; EI MS m/z 310 [MH⁺], FAB MS m/z 310 [MH⁺]; HRMS m/z calcd for C₁₉H₂₄N₃O [MH⁺] 310.1919, found 310.1928.
Preparation of 1-Phenethyl-4-(2-(pyridine-3-yl)ethyl)piperazine

A solution of 1-(4-Phenethylpiperazin-1-yl)-2-(pyridine-3-yl)ethanone (0.75 g, 2.4 mmol) in dry THF (approximately 10 ml) is heated to reflux and 2 M solution of borane-dimethyl sulfide complex in diethyl ether (3.8 ml, 7.7 mmol) is added dropwise over a period of 15 min, allowing dimethyl sulfide to distill off. The reaction mixture is refluxed for about 10 h. The THF solution is then hydrolyzed during addition of 6 N HCl (0.8 ml, 4.8 mmol). After 30 min, the clear solution obtained is cooled to RT and neutralized with 6 N NaOH (1.2 ml, 7.2 mmol). The reaction mixture is stirred at RT for another 1 h. EtOAc (20 ml) is added and the organic layer is washed with aqueous saturated NaHCO₃ (20 ml) and NaCl (20 ml) solution and dried (Na₂SO₄). The solvent is evaporated under reduced pressure to give a product which is further chromatographed on silica (MeOH/EtOAc, 2:10) to give a white solid; yield: 0.31 g, 44% (92% pure by area % HPLC analysis); chemical formula: C₁₉H₂₅N₃; molecular weight: 295.42.

¹H NMR (300 MHz, CDCl₃) δ 2.51-2.89 (16H, m), 7.17-7.29 (5H, m), 7.35-7.39 (1H, m), 7.75 (1H, td, J=7.8 Hz, J=1.7), 8.37 (1H, dd, J=4.9 Hz, J=2.0), 8.44 (1H, d, J=2.0 Hz); FT-IR(NaCl)3450, 2942, 2809, 1652, 1575, 1463, 1421, 1310, 1130, 1007, 851, 700 cm⁻¹; EI MS m/z 295 [M⁺], FAB MS m/z 296 [MH⁺]; HRMS m/z calcd for C₁₉H₂₅N₃ [MH⁺] 295.2048, found 295.2056.
Preparation of 1-Phenethyl-4-(2-(pyridine-3-yl)ethyl)piperazine trihydrobromide

A solution of 1-phenethyl-4-(2-(pyridin-3-yl)ethyl)piperazine (0.23 g, 0.8 mmol) in acetone (approximately 2 ml) is cooled in an ice bath. 1 ml (0.2 g HBr, 2.9 mmol) solution of HBr in ethanol is added dropwise. When precipitation starts, approximately 2 ml of diethyl ether are added. The reaction mixture is stirred in ice for 2 h. The white precipitate is filtered off and successively washed with diethyl ether; yield: 0.246 g, 59% (95% pure by area % HPLC analysis); mp 219-222° C.; chemical formula: C₁₉H₂₈Br₃N₃; molecular weight: 538.16.

¹H NMR (300 MHz, DMSO-d₆) δ 3.03-3.66 (16H, m), 7.24-7.37 (5H, m), 8.02-8.08 (1H, m), 8.44-8.58 (1H, m), 8.86 (1H, m), 8.99 (1H, s); FT-IR (NaCl) 3415, 2228, 2076, 1655, 1557, 1466, 1372, 1249, 1086, 954, 768, 684 cm⁻¹; EI MS m/z 295 [M+], FAB MS m/z 296 [MH⁺]; HRMS m/z calcd for C₁₉H₂₅N₃ [MH⁺] 295.2048, found 295.2043.

### EXAMPLE 3

### 1-Phenethyl-4-(2-(pyridine-4-yl)ethyl)piperazine (LK-9118B)

Preparation of 1-(4-Phenethylpiperazin-1-yl)-2-(pyridine-4-yl)ethanone

To a solution of 1-(2-Phenylethyl)piperazine (0.5 ml, 2.63 mmol) and 4-pyridylacetic acid hydrochloride (0.65 g, 2.81 mmol) in DMF (approximate 5 ml) is added HOBT (0.4 g, 2.81 mmol). The pH of the solution is adjusted to 8 by adding N-methylmorpholine. Coupling reagent EDC (0.55 g, 2.9 mmol) is added. After the reaction mixture is stirred at RT over night, the solvent is evaporated under reduced pressure and the residue is dissolved in EtOAc (15 ml). The organic layer is washed with aqueous saturated NaHCO₃ (15 ml) and NaCl (15 ml) solution and dried (Na₂SO₄). The solvent is evaporated under reduced pressure to give a product which is chromatographed on silica (MeOH/EtOAc, 5:1) to give a yellow oil; yield: 0.81 g, 100%; chemical formula: C₁₉H₂₃N₃O; molecular weight: 309.41.
Preparation of 1-Phenethyl-4-(2-(pyridine-4-yl)ethyl)piperazine

A solution of 1-(4-Phenethylpiperazin-1-yl)-2-(pyridine-4-yl)ethanone (0.844 g, 2.7 mmol) in dry THF (approximately 10 ml) is heated to reflux and a 2 M solution of borane-dimethyl sulfide complex in diethyl ether (4.3 ml, 8.6 mmol) is added dropwise over a period of 15 min, allowing dimethyl sulfide to distill off. The reaction mixture was refluxed for about 10 h. The THF solution is then hydrolyzed during addition of 6 N HCl (1.1 ml, 6.6 mmol). After 30 min, the clear solution obtained is cooled to RT and neutralized with 6 N NaOH (1.7 ml, 10.2 mmol). The reaction mixture is stirred at RT for another 1 h. EtOAc (20 ml) is added and the organic layer is washed with aqueous saturated NaHCO₃ (20 ml) and NaCl (20 ml) solution and dried (Na₂SO₄). The solvent is evaporated under reduced pressure to give a product which is further chromatographed on silica (MeOH/EtOAc, 5:1) to give a white solid; yield: 0.32 g, 40% (97% pure by area % HPLC analysis); chemical formula: C₁₉H₂₅N₃; molecular weight: 295.42.

¹H NMR (300 MHz, DMSO-d₆) δ 2.58-2.65 (12H, m), 2.78-2.84 (4H, m), 7.13-7.32 (7H, m), 8.50 (2H, dd, J=4.4 Hz, J=1.7 Hz); FT-IR (NaCl) 3462, 2947, 2808, 1636, 1600, 1557, 1460, 1315, 1160, 1008, 699 cm⁻¹; EI MS m/z 296 [MH⁺]; HRMS m/z calcd for C₁₉H₂₅N₃ [MH⁺] 296.2127, found 296.2135.

### EXAMPLE 4

### 1-Phenethyl-4-(pyridine-3-ylmethyl)piperazine trihydrobromide (LK-9108)

1-(2-Phenylethyl)piperazine (0.5 ml, 2.6 mmol) and nicotine aldehyde (0.4 ml, 3.9 mmol) are mixed in 1,2-dichloroethane (15 ml) and then treated with NaBH(OAc)₃ (0.74 g, 3.5 mmol). The mixture is stirred at RT under an Ar atmosphere for 2 h. The reaction mixture was quenched by adding aqueous saturated NaHCO₃ (20 ml) solution and the product is extracted with EtOAc (20 ml). The EtOAc extract is dried (NaSO₄) and the solvent is evaporated under reduced pressure to give the crude free base 1-Phenethyl-4-(pyridine-3-ylmethyl)piperazine, which is purified by chromatography on silica (MeOH/EtOAc, 1:5) to give a yellow oil, yield: 0.261 g, 36%.

¹H NMR (300 MHz, CDCl₃) δ 2.54-2.63 (10H, m), 2.78-2.83 (2H, m), 3.54 (2H, s), 7.18-7.31 (6H, m), 7.67 (1H, td, J=7.8 Hz, J=2.0 Hz), 8.50 (1H, dd, J=4.9 Hz, J=1.7 Hz), 8.55 (1H, d, J=1.7 Hz); FT-IR (NaCl) 3354, 2937, 2808, 1576, 1454, 1424, 1349, 1155, 1133, 1009 cm⁻¹; EI MS m/z 281 [M+], FAB MS m/z 282 [MH⁺]; HRMS m/z calcd for C₁₈H₂₃N₃ [M⁺] 281.1892, found 281.1892. The product is converted to the trihydrobromide salt; yield (same procedure as described above): 0.253 g, 91% (99.5% pure by area % HPLC analysis); chemical formula: C₁₈H₂₆Br₃N₃; molecular weight: 524.13.

¹H NMR (300 MHz, DMSO-d₆) δ 3.02-3.71 (12H, m), 4.37 (2H, s), 7.25-7.37 (5H, m), 8.08 (1H, dd, J=7.8 Hz, J=5.6 Hz), 8.66 (1H, td, J=8.1 Hz, J=1.6 Hz), 8.96 (2H, dd, J=5.6 Hz, J=0.98 Hz), 9.06 (1H, d, J=1.5 Hz); FT-IR (NaCl) 3410, 3023, 1627, 1534, 1462, 948, 689 cm⁻¹; FAB MS m/z 282 [MH⁺]; HRMS m/z calcd for C₁₈H₂₃N₃ [M⁺] 281.1892, found 281.1900.

### EXAMPLE 5

### 4-(5-((4-Phenethylpiperazin-1-yl)methyl)pyridine-2-yl)morpholine (LK-9109B)

1-(2-Phenylethyl)piperazine (0.5 g, 2.6 mmol) and 6-Morpholinonicotinaldehyde (0.76 g, 3.9 mmol) are mixed in 1,2-dichloroethane (20 ml) and then treated with NaBH(OAc)₃ (0.87 g, 4.1 mmol). The mixture is stirred at RT under an Ar atmosphere for 2 h. The reaction mixture is quenched by adding aqueous saturated NaHCO₃ (20 ml) solution and the product is extracted with EtOAc (20 ml). The EtOAc extract is dried (NaSO₄) and the solvent is evaporated under reduced pressure to give the crude free base 4-(5-((4-Phenethylpiperazin-1-yl)methyl)pyridine-2-yl)morpholine, which is further purified by chromatography on silica (MeOH/EtOAc, 1:5) to give a yellow crystalline solid, yield: 0.72 g, 74% (99% pure by area % HPLC analysis); chemical formula: C₂₂H₃₀N₄O; molecular weight: 366.5.

¹H NMR (300 MHz, CDCl₃) δ 2.51-2.62 (10H, m), 2.77-2.83 (2H, m), 3.42 (2H, s), 3.49 (4H, t), 3.83 (4H, t), 6.62 (1H, d, J=8.5 Hz), 7.19-7.31 (5H, m), 7.50 (1H, dd, J=8.6 Hz, J=2.4 Hz), 8.10 (1H, d, J=2.0 Hz); FT-IR (NaCl) 3417, 2940, 2815, 1609, 1493, 1448, 1247, 1116, 943, 805, 750, 702, 588 cm⁻¹; EI MS m/z 366 [M⁺]; HRMS m/z calcd for C₂₂H₃₀N₄O [M⁺] 366.2420, found 366.2428.

### EXAMPLE 6

### 1-Phenethyl-4-((6(trifluoromethyl)pyridine-3-yl)methyl)piperazine trihydrobromide (LK-9107)

1-(2-Phenylethyl)piperazine (0.36 ml, 1.69 mmol) and 6-(Trifluoromethyl)nicotinaldehyde (0.5 g, 2.7 mmol) are mixed in 1,2-dichloroethane (15 ml) and then treated with NaBH(OAc)₃ (0.66 g, 3.1 mmol). The mixture is stirred at RT under an Ar atmosphere for 2 h. The reaction mixture is quenched by adding aqueous saturated NaHCO₃ (20 ml) solution and the product is extracted with EtOAc (20 ml). The EtOAc extract is dried (NaSO₄) and the solvent is evaporated under reduced pressure to give the crude free base 1-Phenethyl-4-((6-(trifluoromethyl)pyridine-3-yl)methyl)piperazine, which is further purified by chromatography on silica (MeOH/EtOAc, 1:5) to give a yellow crystalline solid, yield: 0.69 g, 99% (98% pure by area % HPLC analysis); chemical formula: C₁₉H₂₂F₃N₃; molecular weight: 349.39.

¹H NMR (300 MHz, CDCl₃) δ 2.54-2.64 (10H, m), 2.78-2.83 (2H, m), 3.60 (2H, s), 7.19-7.31 (5H, m), 7.65 (1H, d, J=8.1 Hz), 7.87 (1H, dd, J=8.1 Hz, J=1.4 Hz), 8.68 (1H, s); FT-IR (NaCl) 3415, 2933, 2814, 1617, 1496, 1457, 1332, 1129, 1080, 1005, 932, 857, 748, 695, 594 cm⁻¹; EI MS m/z 349 [M⁺], FAB MS m/z 349 [M⁺]; HRMS m/z calcd for C₁₉H₂₂N₃F₃ [M⁺] 349.1766, found 349.1770.

The product is converted to the trihydrobromide salt (same procedure as described above); yield: 0.636 g, 75% (99% pure by area % HPLC analysis); mp 225-228° C.; chemical formula: C₁₉H₂₅Br₃F₃N₃; molecular weight: 592.13.

¹H NMR (300 MHz, DMSO-d₆) δ 3.01-3.73 (14H, m), 7.26-7.38 (5H, m), 8.03 (1H, d, J=7.8 Hz), 8.27 (1H, d, J=7.8 Hz), 8.92 (1H, s); FT-IR (NaCl) 3414, 2974, 2428, 1618, 1452, 1340, 1163, 1086, 949, 697 cm⁻¹; EI MS m/z 349 [M⁺], FAB MS m/z 350 [MH⁺]; HRMS m/z calcd for C₁₉H₂₂N₃F₃ [M⁺] 349.1766, found 349.1770.

### EXAMPLE 7

### 1-(3-Chloro-5-(trifluoromethyl)pyridine-2-yl)-4-phenethyl-1,4-diazepane trihydrobromide (LK-9115)

Preparation of 1-(4-(3-Chloro-5-(trifluoromethyl)pyridine-2-yl)-1,4-diazepan-1-yl)-2-phenylethanone

To a solution of 1-(3-Chloro-5-(trifluoromethyl)pyridine-2-yl)-1,4-diazepane (0.5 g, 1.8 mmol) and 2-Phenylacetic acid (0.25 g, 1.8 mmol) in DMF (approximately 5 ml) is added HOBT (0.25 g, 1.8 mmol). The solution is adjusted to pH 8 by adding N-methylmorpholine. Coupling reagent EDC (0.36 g, 1.9 mmol) is added. After the reaction mixture is stirred at RT over night, the solvent is evaporated under reduced pressure and the residue dissolved in EtOAc (15 ml). The organic layer is washed with aqueous saturated NaHCO₃ (15 ml) and NaCl (15 ml) solution and dried (Na₂SO₄). The solvent is evaporated under reduced pressure to give a product which is further purified by chromatography on silica (MeOH/EtOAc, 5:1) to give a yellow oil; yield: 0.5 g, 70%; chemical formula: C₁₉H₁₉ClF₃N₃O; molecular weight: 397.82.
Preparation of 1-(3-Chloro-5-(trifluoromethyl)pyridine-2-yl)-4-phenethyl-1,4-diazepane

A solution of 1-(4-(3-Chloro-5-(trifluoromethyl)pyridine-2-yl)-1,4-diazepan-1-yl)-2-phenylethanone (0.5 g, 1.3 mmol) in dry THF (approximately 6 ml) is heated to reflux and 2 M solution of borane-dimethyl sulfide complex in diethyl ether (2.5 ml, 5.1 mmol) is added in drops over the period of 15 min, allowing dimethyl sulfide to distill off. The reaction mixture is refluxed for about 10 h. The THF solution is then hydrolyzed during addition of 6 N HCl (0.5 ml, 3.0 mmol). After 30 min, the clear solution obtained is cooled to RT and neutralized with 6 N NaOH (0.8 ml, 4.8 mmol). The reaction mixture is stirred at RT for another 1 h. EtOAc (15 ml) is added and the organic layer is washed with aqueous saturated NaHCO₃ (15 ml) and NaCl (15 ml) solution and dried (Na₂SO₄). The solvent is evaporated under reduced pressure to give a product which is further purified by chromatography on silica (MeOH/EtOAc, 1:50) to give a pale yellow oil; yield: 0.32 g, 66%; chemical formula: C₁₉H₂₁ClF₃N₃; molecular weight: 383.84.

¹H NMR (300 MHz, CDCl₃) δ 2.02-2.08 (2H, m), 2.75-2.96 (8H, m), 3.79-3.87 (4H, m), 7.18-7.32 (5H, m), 7.62-7.69 (1H, m), 8.29-8.31 (1H, m); FT-IR (NaCl) 2942, 1606, 1498, 1410, 1317, 1120, 912 cm⁻¹; EI MS m/z 384 [MH⁺]; HRMS m/z calcd for C₁₉H₂₂N₃F₃Cl₃ [MH⁺] 384.1454, found 384.1462.

The product is converted to the trihydrobromide salt (same procedure as described above); yield: 0.26 g, 66% (99% pure by area % HPLC analysis); mp 175-177° C.; chemical formula: C₁₉H₂₄Br₃ClF₃N₃; molecular weight: 626.57.

¹H NMR (300 MHz, DMSO-d₆) δ 3.05-3.91 (14H, m), 7.22-7.37 (5H, m), 8.14 (1H, d, J=Hz), 8.48 (1H, m); FT-IR (NaCl) 3412, 2948, 2727, 1639, 1596, 1442, 1287, 1173, 1139, 701 cm⁻¹; EI MS m/z 383 [M⁺], FAB MS m/z 384 [MH⁺]; HRMS m/z calcd for C₁₉H₂₁N₃F₃Cl₃ [M⁺] 383.1376, found 383.1386.

### EXAMPLE 8

### 1-(Naphthalen-2-yl)-2-(4-phenethylpiperazin-1-yl)ethanol (LK-9111B)

To a solution of 2-Bromo-1-(naphthalen-2-yl)ethanone (0.5 g, 2.0 mmol) in anhydrous ethanol (15 ml) is added NaBH₄ (0.28 g, 7.4 mmol). The reaction mixture is stirred at RT for 2 h. The mixture is filtered and 1-(2-Phenylethyl)piperazine (0.9 ml, 4.7 mmol) is added to the filtrate. The solution is heated to reflux and refluxed for 5 h. Excessive ethanol is removed by distillation. The residue is dissolved in chloroform (20 ml), the insoluble parts are filtered off and the filtrate is concentrated by distillation under reduced pressure. The product is further purified by chromatography on silica (MeOH/EtOAc, 5:1) to give a white solid; yield: 0.174 g, 32% (96% pure by area % HPLC analysis); mp 126-129° C.; chemical formula: C₂₄H₂₈N₂O; molecular weight: 360.49.

¹H NMR (300 MHz, CDCl₃) δ 2.60-2.93 (14H, m), 4.98 (1H, dd, J=9.8 Hz, J=3.9 Hz), 7.17-7.82 (12H, m); FT-IR (NaCl) 3410, 2822, 1642, 1452, 1328, 1165, 1120, 1070, 1005, 744, 698 cm⁻¹; EI MS m/z 359 [M−1]⁺; HRMS m/z calcd for C₂₄H₂₇N₂O [M−1]+359.2123, found 359.2125.

### EXAMPLE 9

### 2-(4-Phenethylpiperazine-1-yl)-1-(pyridine-4-yl)ethanol (LK-9110B)

Preparation of 2-bromo-1-(4-pyridinyl)-1-ethanone hydrobromide as starting material is exemplary published in e.g. WO 2004/007456.

To a solution of 2-Bromo-1-(4-pyridinyl)-1-ethanone hydrobromide (4.0 g, 14.4 mmol) in anhydrous ethanol (80 ml) is added NaBH (2.0 g, 52.8 mmol). The reaction mixture is stirred at RT for 2 h. The mixture is filtered and 1-(2-Phenylethyl)piperazine (4.9 ml, 26.0 mmol) is added to the filtrate. The solution is heated to reflux and refluxed for 5 h. Excessive ethanol is removed by distillation. The resulting pale yellow solid is dissolved in chloroform (80 ml), the insoluble parts are filtered off and the filtrate is concentrated by distillation under reduced pressure. The product is purified by chromatography on silica (MeOH/EtOAc, 5:1) to give a pale yellow solid; yield: 32%; chemical formula: C₁₉H₂₅N₃O; molecular weight: 311.42.

¹H NMR (300 MHz, CDCl₃) δ 2.83-2.84 (m, 14H), 4.73 (dd, 1H, J=10.5 Hz, J=3.8 Hz), 7.20-7.30 (m, 5H), 7.31 (dd, 2H, J=4.7 Hz, J=1.5 Hz), 8.57 (dd, 2H, J=4.4 Hz, J=1.5 Hz);

FT-IR (KBr) 3420, 1639, 1458, 1409, 1128, 854, 696, 622 cm⁻¹;

EI MS m/z 310 [M−H+];

HR MS m/z calcd for C₁₉H₂₄N₃O [M−H+] C₁₉H₂₄N₃O 310.191938, found 310.192050.

### EXAMPLE 10

### 1-Phenethyl-4-(3-(pyridine-3-yl)propyl)piperazine trihydrobromide (LK-9140)

Preparation of 1-(4-Phenethylpiperazin-1-yl)-3-(pyridine-3-yl)propanone

To a solution of 1-(2-Phenylethyl)piperazine (0.50 g, 2.63 mmol) and 3-Pyridylpropionic acid hydrochloride (0.40 g, 2.63 mmol) in DMF (approximately 5 ml) is added HOBT (0.42 g, 3.14 mmol). The solution is adjusted to pH 8 by adding N-methylmorpholine. Coupling reagent EDC (0.63 g, 3.25 mmol) is added. After the reaction mixture is stirred at RT over night, the solvent is evaporated under reduced pressure and the residue dissolved in EtOAc (20 ml). The organic layer is washed with aqueous saturated NaHCO₃ (2×20 ml) and NaCl (20 ml) solution and dried (Na₂SO₄). The solvent is evaporated under reduced pressure to give a product which is purified by chromatography on silica (MeOH/EtOAc, 5:1) to give a brownish solid; yield: 0.69 g, 81%; chemical formula: C₂₀H₂₅N₃O; molecular weight: 323.21.

¹H NMR (300 MHz, CDCl₃) δ 2.35 (t, 4H), 2.48-2.54 (m, 6H), 2.61-2.74 (m, 4H), 2.78-2.85 (t, 2H, J=7.4 Hz), 7.17-7.25 (m, 5H), 7.30 (ddd, 1H, J=7.8 Hz, J=4.8 Hz, J=1.9 Hz), 7.66 (ddd, 1H, J=7.8 Hz, J=1.9 Hz, J=1.9 Hz), 8.38 (dd, 1H, J=4.8 Hz, J=1.6 Hz), 8.46 (d, 1H, J=2.1 Hz);

FT-IR (NaCl) 3026, 2933, 2809, 1644, 1440, 1133, 1001, 702 cm⁻¹;

FAB MS m/z 324 [MH⁺]; HR MS m/z calcd for C₂₀H₂₆N₃O: 324.207588; found: 324.208350
Preparation of 1-Phenethyl-4-(2-(pyridine-3-yl)propyl)piperazine

A solution of 1-(4-Phenethylpiperazin-1-yl)-2-(pyridine-3-yl)propanone (0.69 g 2.10 mmol) in dry THF (approximately 10 ml) is heated to reflux and 2 M solution of borane-dimethyl sulfide complex in diethyl ether (3.29 ml, 6.57 mmol) is added dropwise over the period of 15 min allowing dimethyl sulfide to distill off. The reaction mixture is refluxed for about 10 h. The THF solution was then hydrolyzed during addition of 6 N HCl (0.69 ml. 4.10 mmol). After 30 min the clear solution obtained is cooled to RT and neutralized with 6 N NaOH (1.04 ml, 6.18 mmol). The reaction mixture is stirred at RT for another 1 h. EtOAc (20 ml) is added and the organic layer is washed with aqueous saturated NaHCO₃ (20 ml) and NaCl (20 ml) solution and dried (Na₂SO₄). The solvent is evaporated under reduced pressure to give a product which is further purified by chromatography on silica (MeOH/EtOAc, 5:1) to give a white solid; yield: 0.30 g, 46%; chemical formula: C₂₀H₂₇N₃; molecular weight: 309.22. ¹H NMR (300 MHz, CDCl₃) δ 1.83 (q, 2H, J=7.6 Hz), 2.38 (t, 2H, J=7.5 Hz), 2.52-2.61 (m, 10H), 2.65 (t, 1H, J=7.2 Hz), 2.81 (t, 2H, J=8.1 Hz), 7.18-7.27 (m, 5H), 7.27-7.31 (m, 1H), 7.51 (ddd, 1H, J=7.8 Hz, J=2.1 Hz, J=1.8 Hz), 8.44 (dd, 1H, J=4.8 Hz, J=1.8 Hz), 8.46 (d, 1H, J=2.1 Hz);

FT-IR (KBr) 3388, 3025, 2941, 2809, 1672, 1574, 1453, 1270, 1132, 1009, 700 cm⁻¹

EI MS m/z 309 [M⁺];

HR MS m/z calcd for C₂₀H₂₇N₃: 309.220498; found: 309.220800

The product is converted to the trihydrobromide salt (same procedure as described above); yield: 0.41 g, 91% (98.8% pure by area % HPLC analysis); mp 262-264° C.; chemical formula: C₂₀H₃₀N₃Br₃; molecular weight: 552.18.

¹H NMR (300 MHz, DMSO-d₆) δ 2.11 (q, 2H, J=7.8 Hz), 2.91 (t, 2H, J=7.5 Hz), 3.06 (t, 2H, J=8.4 Hz), 3.27 (t, 2H, J=7.5 Hz), 3.47 (t, 2H, J=8.4 Hz), 3.56-3.66 (m, 8H), 7.27-7.36 (m, 5H), 8.04 (dd, 1H, J=8.1 Hz, J=5.7 Hz), 8.53 (d, 1H, J=8.4 Hz), 8.85 (d, 1H, J=5.4 Hz), 8.92-8.94 (m, 1H);

FT-IR (KBr) 3438, 2977, 2548, 2054, 1944, 1613, 1556, 1468, 1368, 1264, 1019, 911, 804, 755, 686 cm⁻¹;

EI MS m/z 309 [M⁺];

### EXAMPLE 11

### 2-(4-(4-Fluorophenyl)piperazine-1-yl)-1-(pyridine-3-yl)ethanol trihydrobromide (LK-9144)

Preparation of 2-(4-(4-Fluorophenyl)piperazine-1-yl)-1-(pyridine-3-yl)ethanol

Same procedure as described for LK-980 and LK-9110B.

yield: 72% (95.6% pure by area % HPLC analysis); mp 66-68° C.; chemical formula:

C₁₇H₂₀N₃° F.; molecular weight: 301.16;

¹H NMR (300 MHz, DMSO-d₆) δ 2.51 (d, 2H, J=4.8 Hz), 2.57-2.66 (m, 4H), 3.04 (t, 4H, J=4.8 Hz), 4.81 (m, 1H), 5.30 (d, 1H, J=3.9 Hz), 6.91-6.95 (m, 2H), 7.00-7.06 (m, 2H), 7.35 (dd, 1H, J=7.6 Hz, J=5.4 Hz), 7.77 (ddd, 1H, J=8.1 Hz, J=1.8 Hz, J=1.8 Hz), 8.45 (dd, 1H, J=4.6 Hz, J=1.6 Hz), 8.57 (d, 1H, J=1.6 Hz);

FTIR (KBr) 3354, 1579, 1510, 1425, 1235, 1144, 898, 817, 715 cm⁻¹;

EI MS m/z 301 [M⁺];

HR MS m/z calcd for C₁₇H₂₀N₃° F.: 301.159041; found: 301.160120;
Preparation of 2-(4-(4-Fluorophenyl)piperazine-1-yl)-1-(pyridine-3-yl)ethanol trihydrobromide

Same procedure as described for LK-980

yield: 89% (98.9% pure by area % HPLC analysis); mp 198-201° C.; chemical formula: C₁₇H₂₃N₃OFBr₃; molecular weight: 544.08;

¹H NMR (300 MHz, DMSO-d₆) δ 3.12-3.30 (m, 4H), 3.42-3.50 (m, 2H), 3.64-3.74 (m, 4H), 4.96 (t, 1H, J=4.2 Hz), 5.52-5.65 (m, 1H), 7.01-7.13 (m, 4H), 8.16 (dd, 1H, J=8.0 Hz), 8.71 (ddd, 1H, J=8.4 Hz, J=1.5 Hz, J=1.5 Hz), 8.96 (dd, 1H, J=5.7 Hz), 9.05 (d, 1H, J=1.5 Hz);

FTIR (KBr) 3259, 2355, 1632, 1556, 1509, 1353, 1244, 1158, 1025, 973, 840, 684, 536 cm⁻¹; EI MS m/z 301 [M⁺];

### EXAMPLE 12

### 2-(4-(4-Fluorobenzyl)piperazine-1-yl)-1-(pyridine-3-yl)ethanol trihydrobromide (LK-9148)

Preparation of 2-(4-(4-Fluorobenzyl)piperazine-1-yl)-1-(pyridine-3-yl)ethanol

Same procedure as described for LK-980
Preparation of 2-(4-(4-Fluorobenzyl)piperazine-1-yl)-1-(pyridine-3-yl)ethanol trihydrobromide

Same procedure as described for LK-980 trihydrobromide.

yield: 89% (99.7% pure by area % HPLC analysis); mp 220-223° C.; chemical formula: C₁₈H₂₅N₃OFBr₃; molecular weight: 558.11;

¹H NMR (300 MHz, DMSO-d₆) δ 3.52-3.78 (m, br, 12H), 4.45-4.48 (m, 1H), 5.34-5.39 (m, 1H), 7.31-7.37 (m, 2H), 7.64-7.69 (m, 2H), 8.04 (dd, 1H, J=8.1 Hz, J=5.6 Hz), 8.58 (ddd, 1H, J=8.1 Hz, J=1.6 Hz, J=1.6 Hz), 8.87 (dd, 1H, J=5.7 Hz, J=1.6 Hz), 8.97 (d, 1H, J=1.6 Hz);

FTIR (KBr) 3258, 2520, 1606, 1516, 1436, 1233, 1070, 844, 683, 609 cm⁻¹;

FAB MS m/z 316 [MH⁺];

### EXAMPLE 13

### 2-(4-Phenethylpiperazine-1-yl)-1-(6-methylpyridin-3-yl)ethanol trihydrobromide (LK-9139)

Preparation of 2-(4-Phenethylpiperazine-1-yl)-1-(6-methylpyridin-3-yl)ethanol
Preparation of 2-(4-Phenethylpiperazine-1-yl)-1-(6-methylpyridin-3-yl)ethanol trihydrobromide

Same procedure as above.

yield: 86% (97.0% pure by area % HPLC analysis); mp 254-256° C.; chemical formula: C₁₉H₂₈N₃Br₃; molecular weight: 538.14;

¹H NMR (300 MHz, DMSO-d₆) δ 2.75 (s, 3H), 3.04-3.09 (m, 4H), 3.35-3.46 (m, 8H), 4.24 (s, 2H), 7.27-7.38 (m, 5H), 7.99 (d, 1H, J=8.1 Hz), 8.57 (dd, 1H, J=8.1 Hz, J=1.6 Hz), 8.94 (d, 1H, J=1.6 Hz);

FTIR (KBr) 3426, 2974, 2534, 1645, 1614, 1456, 1294, 1150, 952, 753, 701 cm⁻¹;

EI MS m/z 295 [M⁺];

### EXAMPLE 14

### 1-(3,4-di-Fluorophenethyl)-4-(2-(pyridin-4-yl)ethyl)piperazine trihydrobromide (LK-9131)

Preparation of 1-(4-(3,4-Difluorophenethyl)piperazin-1-yl)-2-(pyridin-4-yl)ethanone
Preparation of 1-(3,4-Difluorophenethyl)-4-(2-(pyridin-4-yl)ethyl)piperazine
Preparation of 1-(3,4-Difluorophenethyl)-4-(2-(pyridin-4-yl)ethyl)piperazine trihydrobromide

Same procedure as above.

yield: 40% (% pure by area % HPLC analysis); mp 256-259° C.; chemical formula: C₁₉H₂₆N₃F₂Br₃; molecular weight: 574.13;

¹H NMR (300 MHz, CDCl₃) δ 2.97-3.36 (m, 16H), 7.13-7.45 (m, 3H), 7.91 (d, 2H, J=6.6 Hz), 8.80 (dd, 2H, J=5.4 Hz, J=1.2 Hz);

FTIR (KBr) 2977, 2531, 2436, 1603, 1518, 1441, 1282, 57, 806 cm⁻¹;

EI MS m/z 331 [M⁺]

### EXAMPLE 15

### 1-(3,4-Dichlorophenethyl)-4-(2-(pyridin-2-yl)ethyl)piperazine trihydrobromide (LK-9137)

Preparation of 1-(4-(3,4-Dichlorophenethyl)piperazin-1-yl)-2-(pyridin-2-yl)ethanone
Preparation of 1-(3,4-Dichlorophenethyl)-4-(2-(pyridin-2-yl)ethyl)piperazine
Preparation of 1-(3,4-Dichlorophenethyl)-4-(2-(pyridin-2-yl)ethyl)piperazine trihydrobromide

Same procedure as above.

yield: 89% (99.9% pure by area % HPLC analysis); mp 207-209° C.; chemical formula: C₁₉H₂₆N₃Cl₂Br₃; molecular weight: 607.03;

¹H NMR (300 MHz, DMSO-d₆) δ 3.08 (t, 2H, J=8.1 Hz), 3.42-3.54 (m, 12H), 3.60 (t, 2H, J=7.5 Hz), 7.33 (dd, 1H, J=8.1 Hz, J=2.1 Hz), 7.62 (d, 1H, J=8.1 Hz), 7.65 (d, 1H, J=2.1 Hz), 7.81 (ddd, 1H, J=6.3 Hz, J=6.3 Hz, J=0.9 Hz), 7.93 (d, 1H, J=7.8 Hz), 8.38 (ddd, 1H, J=7.5 Hz, J=7.5 Hz, J=1.2 Hz), 8.82 (dd, 1H, J=5.4 Hz, J=0.9 Hz);

FTIR (KBr) 3509, 2979, 2646, 2446, 1634, 1468, 1272, 958, 771 cm⁻¹;

EI MS m/z 364 [MH⁺];

### EXAMPLE 16

### 1-(3,4-Difluorophenethyl)-4-(2-(pyridin-2-yl)ethyl)piperazine trihydrobromide (LK-9138)

Preparation of 1-(4-(3,4-Difluorophenethyl)piperazin-1-yl)-2-(pyridin-2-yl)ethanone
Preparation of 1-(3,4-Difluorophenethyl)-4-(2-(pyridin-2-yl)ethyl)piperazine
Preparation of 1-(3,4-Difluorophenethyl)-4-(2-(pyridin-2-yl)ethyl)piperazine trihydrobromide

Same procedure as above.

yield: 83% (100.0% pure by area % HPLC analysis); mp 212-213° C.; chemical formula: C₁₉H₂₆N₃F₂Br₃; molecular weight: 574.13;

¹H NMR (300 MHz, DMSO-d₆) δ 3.09 (t, 2H, J=8.2 Hz), 3.47-3.80 (m, b, 12H), 3.63 (t, 2H, J=7.4 Hz), 7.16-7.20 (m, 1H), 7.38-7.50 (m, 2H), 7.82 (dd, 1H, J=6.6 Hz, J=6.6 Hz), 7.94 (d, 1H, J=8.1 Hz), 8.39 (dd, 1H, J=7.8 Hz, J=7.8 Hz), 8.83 (d, 1H, J=5.7 Hz);

FTIR (KBr) 3494, 2975, 2558, 1619, 1518, 1468, 1278, 962, 776 cm⁻¹;

EI MS m/z 332 [MH⁺]

### EXAMPLE 17

### 1-(3,4-Dichlorophenethyl)-4-(2-(pyridin-4-yl)ethyl)piperazine trihydrobromide (LK-9135)

Preparation of 1-(4-(3,4-Dichlorophenethyl)piperazin-1-yl)-2-(pyridin-4-yl)ethanone
Preparation of 1-(3,4-Dichlorophenethyl)-4-(2-(pyridin-4-yl)ethyl)piperazine
Preparation of 1-(3,4-Dichlorophenethyl)-4-(2-(pyridin-4-yl)ethyl)piperazine trihydrobromide

Same procedure as above.

yield: 73% (98.6% pure by area % HPLC analysis); mp 253-255° C.; chemical formula: C₁₉H₂₆N₃Cl₂Br₃; molecular weight: 607.03;

¹H NMR (300 MHz, DMSO-d₆) δ 3.03 (t, 4H, J=8.1 Hz), 3.23-3.55 (m, 12H), 7.31 (dd, 1H, J=8.1 Hz, J=1.8 Hz), 7.61 (d, 1H, J=8.1 Hz), 7.62 (d, 1H, J=2.1 Hz), 7.95 (d, 2H, J=6.3 Hz), 8.84 (d, 2H, J=6.3 Hz);

FTIR (KBr) 3472, 2974, 2394, 1632, 1588, 1499, 1187, 957, 799 cm⁻¹;

FAB MS m/z 364 [MH⁺];

### EXAMPLE 18

### 1-((6-Methoxypyridin-3-yl)methyl)-4-phenethylpiperazine trihydrobromide (LK 9106)

Same procedure as for LK-9107

Yield: 11%; mp 215-217° C. and 251-253° C. (two isomers); chemical formula: C₁₉H₂₈Br₃N₃O; molecular weight: 554.16;

¹H NMR (300 MHz, DMSO-d₆) 2.96-3.93 (m, 17H), 6.35-6.87 (m, 1H), 7.23-7.35 (m, 5H), 7.53 (d, 1H, J=8.2 Hz), 7.80-8.23 (s, 1H).

FTIR (KBr) 3415, 2539, 1642, 1612, 1558, 1331, 1299, 1165, 955, 756 cm⁻¹;

FAB MS m/z 312 [MH⁺];

### EXAMPLE 19

The inhibitory action on cholesterol biosynthesis of the compounds of the invention is determined as follows.

An ex vivo method of metabolic labeling of immortal human hepatocytes is employed. The radioactively labeled early precursor of cholesterol [3H]acetate is added to cells with or without addition of an active compound.

### Materials and Methods

Cell culture incubation with potential inhibitors of cholesterol biosynthesis and statins Human hepatoma cell line (HepG₂-ATCC No. HB-8065) is split in the recommended ratio (1:2-3) into 75 cm² cell flasks using two flasks per experimental condition. Cells are incubated at 37° C. with 5% CO₂ in Dulbeco's modified Eagle medium (DMEM high (Sigma)) containing 5% calf serum (Sigma) and 1% L-glutamine (Sigma). After 24-hours-culturing in the growth medium, the medium is replaced with the one supplemented with 10 μM concentration of a LK compound, potential inhibitor of cholesterol biosynthesis. 10 μM solution of atorvastatin, inhibitor of HMG-CoA reductase, serves as a positive control.

After 24 hours the growth medium is changed and [³H]acetate (NEN™ Life Science Products) added in a concentration of 40 μCi per ml of the medium (400 μCi per flask). After 4 hours cells are harvested using 2 ml of trypsin (Sigma) and the cell pellet is resuspended in 1 ml of distilled water. Cells are homogenized using the freeze and thaw method. From cell homogenate sterols are extracted and protein concentration determined using the Bio-Rad reagent.

Sterol extraction

Homogenates are transferred into 4 ml glass vial and sterols are extracted in 3 ml of extraction solution (75% n-heptane: 25% isopropanol (vol./vol.)) with ergosterol as an internal standard. For better extraction 100 μl of 0.3M NaH₂PO₄ (pH=1.0) is added. Closed vials are vigorously shaken (1800 rpm) for 2 hours. After extraction procedure vials are centrifuged (2000 g, 10 min) and the organic phase is transferred to fresh conical glass tubes. Extraction procedure is repeated using 1 ml of extraction solution for 15 min. Organic phases are pooled. Primary extracts are dried in vacuum centrifuge, redissolved in 3 ml of n-heptane, and incubated for 10 min at room temperature with mild shaking. After centrifugation (10 min, 2000 g) extracts are transferred into fresh glass tubes and stored in dark and cold.

### HPLC Analysis

The organic phase is dried, reconstituted in mobile phase for reversed phase HPLC separation and loaded onto a-HPLC column, Prism-RPN, 5 μm, 250×4,6 mm running in 100% acetonitrile at 1.00 ml/min at 40° C. temperature. Scintillation liquid is added after UV detection at 30 ml/h, at room temperature, to evaluate tritium labeled sterols on a radio detector.

### Sterol Detection

Sterols are determined by comparing the eluted peaks with runs of commercial standards of lanosterol, ergosterol, desmosterol, zymosterol, 7-dehydrocholesterol, lathosterol and cholesterol or laboratory standards (FF-MAS-4,4-Dimethylcholesta-8(9),14,24-triene-3β-ol- and T-MAS-4,4-Dimethylcholesta8(9),24,diene-3,6-ol, Laboratory of Reproductive Biology, The Juliane Marie Center for Children, Women and Reproduction, University Hospital of Copenhagen, DK-2100 Copenhagen, Denmark). Results were normalized on ergosterol quantity and protein concentration. Results are presented as a mean from 4 separated experiments.

### Results

The influence of a 10 μM solution of novel LK-substances on cholesterol biosynthesis in the assay on HepG2 cells is presented in Table I. Compounds are arranged according to the building blocks, three of them are 2-pyridineethanols, 2-pyridineethylpiperazines and pyridinemethylpiperazines.

Normal medium pertains to cell cultures without a compound of the invention. Its sterol levels were used for normalization. 10 μM solution of atorvastatin serves as a positive control. At the level of radio-HPLC chromatograms, atorvastatin control shows complete suppression of cholesterol biosynthesis as well as cholesterol precursors—desmosterol, zymosterol +7-dehydrocholesterol, lathosterol, FF-MAS and lanosterol—determined in the assay.

All novel compounds, at a concentration of 10 μM, almost completely block de novo synthesized cholesterol in cell cultures. Most compounds block desmosterol, too. Compounds could be classified with regard to sterol profiles as presented in Table II. First group is relatively weaker in cholesterol and desmosterol inhibition, second group has characteristic FF-MAS accumulation, third group potently blocks all sterols up to FF-MAS. Up and down regulation is exerted for zymosterol and 7-dehydrocholesterol, lathosterol is down-regulated. Some compounds such as LK-9109B and LK-911 lB notably accumulate FF-MAS. Lanosterol is close to unity for most compounds, only LK-911 lB indicates lanosterol accumulation. LK-9140, 9138, 9135 and LK-9131 reduce all sterol levels below unity.

Preferred sterol profile for novel compounds of this invention is an inhibition of cholesterol biosynthesis at 10 μM concentration leading to lowered cholesterol and desmosterol level and no accumulation of lanosterol and other post-lanosterol intermediates.

TABLE I
Compounds arranged according to synthetic building blocks

| | | | zymosterol+7- | | | |
|---|---|---|---|---|---|---|
| | | | dehydrochole- | | | |
| | cholesterol | desmosterol | sterol | lathosterol | FF-MAS | lanosterol |
| normal medium | 1.00 | 1.00 | 1.00 | 1.00 | 1.00 | 1.00 |
| atorvastatin | 0.00 | 0.00 | 0.00 | 0.00 | 0.00 | 0.00 |
| LK-980 | 0.05 | 0.00 | 1.02 | 0.49 | 2.54 | 0.96 |
| LK-9110B | 0.00 | 0.17 | 1.37 | 0.78 | 3.74 | 0.96 |
| LK-9144 | 0.09 | 0.05 | 1.50 | 0.56 | 0.99 | 0.88 |
| LK-9148 | 0.14 | 0.38 | 1.44 | 0.65 | 1.25 | 0.80 |
| LK-9139 | 0.01 | 0.14 | 0.38 | 0.15 | 1.19 | 0.38 |
| LK-9100 | 0.00 | 0.00 | 0.74 | 0.30 | 2.41 | 0.83 |
| LK-9101B | 0.12 | 0.10 | 1.55 | 0.54 | 1.36 | 0.94 |
| LK-9118B | 0.00 | 0.47 | 0.90 | 0.23 | 3.50 | 1.46 |
| LK-9135 | 0.00 | 0.00 | 0.02 | 0.00 | 0.27 | 0.43 |
| LK-9137 | 0.06 | 0.22 | 1.41 | 0.60 | 1.42 | 0.63 |
| LK-9138 | 0.01 | 0.07 | 0.21 | 0.08 | 0.70 | 0.25 |
| LK-9131 | 0.00 | 0.01 | 0.09 | 0.05 | 0.72 | 0.69 |
| LK-9106 | 0.00 | 0.00 | 0.46 | 0.31 | 2.00 | 0.52 |
| LK-9107 | 0.00 | 0.00 | 0.20 | 0.11 | 1.29 | 0.36 |
| LK-9108 | 0.00 | 0.00 | 1.40 | 0.74 | 3.10 | 1.10 |
| LK-9109B | 0.00 | 0.29 | 1.00 | 0.09 | 6.50 | 0.95 |
| LK-9111B | 0.00 | 0.00 | 1.71 | 0.46 | 9.01 | 1.97 |
| LK-9115 | 0.00 | 0.00 | 0.91 | 0.21 | 4.42 | 0.84 |
| LK-9140 | 0.00 | 0.01 | 0.15 | 0.03 | 0.72 | 0.24 |

TABLE II
Compounds arranged according to the sterol profile

| | | | zymosterol+7- | | | |
|---|---|---|---|---|---|---|
| | | | dehydrochole- | | | |
| | cholesterol | desmosterol | sterol | lathosterol | FF-MAS | lanosterol |
| normal medium | 1.00 | 1.00 | 1.00 | 1.00 | 1.00 | 1.00 |
| atorvastatin | 0.00 | 0.00 | 0.00 | 0.00 | 0.00 | 0.00 |
| LK-9144 | 0.09 | 0.05 | 1.50 | 0.56 | 0.99 | 0.88 |
| LK-9148 | 0.14 | 0.38 | 1.44 | 0.65 | 1.25 | 0.80 |
| LK-9101B | 0.12 | 0.10 | 1.55 | 0.54 | 1.36 | 0.94 |
| LK-9137 | 0.06 | 0.22 | 1.41 | 0.60 | 1.42 | 0.63 |
| LK-9100 | 0.00 | 0.00 | 0.74 | 0.30 | 2.41 | 0.83 |
| LK-980 | 0.05 | 0.00 | 1.02 | 0.49 | 2.54 | 0.96 |
| LK-9115 | 0.00 | 0.00 | 0.91 | 0.21 | 4.42 | 0.84 |
| LK-9118B | 0.00 | 0.47 | 0.90 | 0.23 | 3.50 | 1.46 |
| LK-9109B | 0.00 | 0.29 | 1.00 | 0.09 | 6.50 | 0.95 |
| LK-9108 | 0.00 | 0.00 | 1.40 | 0.74 | 3.10 | 1.10 |
| LK-9110B | 0.00 | 0.17 | 1.37 | 0.78 | 3.74 | 0.96 |
| LK-9111B | 0.00 | 0.00 | 1.71 | 0.46 | 9.01 | 1.97 |
| LK-9106 | 0.00 | 0.00 | 0.46 | 0.31 | 2.00 | 0.52 |
| LK-9107 | 0.00 | 0.00 | 0.20 | 0.11 | 1.29 | 0.36 |
| LK-9139 | 0.01 | 0.14 | 0.38 | 0.15 | 1.19 | 0.38 |
| LK-9135 | 0.00 | 0.00 | 0.02 | 0.00 | 0.27 | 0.43 |
| LK-9138 | 0.01 | 0.07 | 0.21 | 0.08 | 0.70 | 0.25 |
| LK-9131 | 0.00 | 0.01 | 0.09 | 0.05 | 0.72 | 0.69 |
| LK-9140 | 0.00 | 0.01 | 0.15 | 0.03 | 0.72 | 0.24 |

1-12. (canceled)

13. A compound of formula I 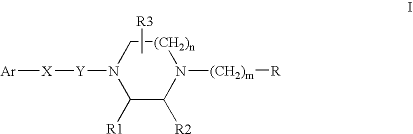
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group.

14. A compound according to claim 13, wherein Ar is pyridyl and R is phenyl.

15. A compound according to claim 13, wherein the compound is selected from the group consisting of
2-(4-Phenethylpiperazin-1-yl)-1-(pyridine-3-yl)ethanol;
1-Phenethyl-4-(2-(pyridine-3-yl)ethyl)piperazine;
1-Phenethyl-4-(2-(pyridine-4-yl)ethyl)piperazine;
1-Phenethyl-4-(pyridine-3-ylmethyl)piperazine;
4-(5-((4-Phenethylpiperazin-1-yl)methyl)pyridine-2-yl)morpholine;
1-Phenethyl-4-((6-(trifluoromethyl)pyridine-3-yl)methyl)piperazine;
1-(3-Chloro-5-(trifluoromethyl)pyridine-2-yl)-4-phenethyl-1,4-diazepane;
1-(Naphthalen-2-yl)-2-(4-phenethylpiperazin-1-yl)ethanol;
2-(4-Phenethylpiperazine-1-yl)-1-(pyridine-4-yl)ethanol;
1-Phenethyl-4-(3-(pyridine-3-yl)propyl)piperazine;
2-(4-(4-Fluorophenyl)piperazine-1-yl)-1-(pyridine-3-yl)ethanol;
2-(4-(4-Fluorobenzyl)piperazine-1-yl)-1-(pyridine-3-yl)ethanol;
2-(4-Phenethylpiperazine-1-yl)-1-(6-methylpyridin-3-yl)ethanol;
1-(3,4-di-Fluorophenethyl)-4-(2-(pyridine-4-yl)ethyl)piperazine;
1-(3,4-Dichlorophenethyl)-4-(2-(pyridine-2-yl)ethyl)piperazine;
1-(3,4-Difluorophenethyl)-4-(2-(pyridine-2-yl)ethyl)piperazine;
1-(3,4-Dichlorophenethyl)-4-(2-(pyridine-4-yl)ethyl)piperazine;
1-Phenethyl-4-(2-(piperidin-3-yl)ethyl)piperazine; and
1-((6-Methoxypyridin-3-yl)methyl)-4-phenethylpiperazine.

16. A compound of claim 13, wherein the compound is in the form of a salt.

17. A method comprising using a compound of formula I as a pharmaceutical, wherein the compound of formula I comprises 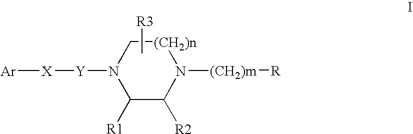
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group.

18. A method of treating hypercholesterolemia and hyperlipidemia in a subject, wherein an effective amount of a compound of formula I is administered to the subject: 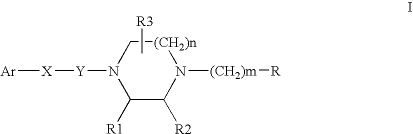
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C-6 alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group.

19. A pharmaceutical composition comprising a compound of formula I and at least one pharmaceutical excipient, wherein the compound of formula I comprises 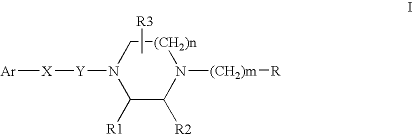
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₄ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group.

20. A pharmaceutical composition according to claim 19, further comprising at least one other pharmaceutically active agent.

21. A process for the production of arylethanol or heteroarylamines of formula I 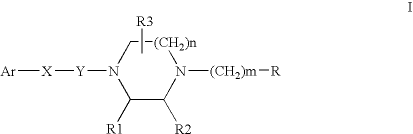
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
the method comprising the steps of alkylating cyclic secondary amines of formula XI wherein n, m, R₁, R₂, R₃ and R are defined above, 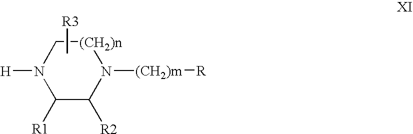
with aryloxirane, heteroaryloxirane of formula XII wherein Ar is as defined above, 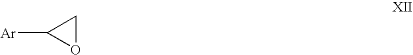
to arylethanol amines or heteroarylethanol amines of formula I and optionally converting them into the physiologically acceptable acid addition salts thereof.

22. A process for the production of Ar-alkyl compounds of formula I 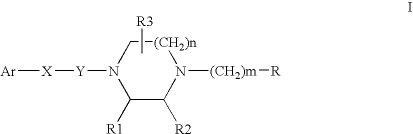
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
the method comprising the steps of coupling cyclic secondary amines of formula XI wherein n, m, R₁, R₂, R₃ and R are as defined above, 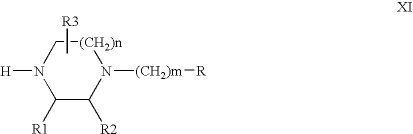
with carboxylic acid derivatives of formula XIII
Ar—X—COOH XIII
wherein Ar and X are as defined above to compounds of formula XIV 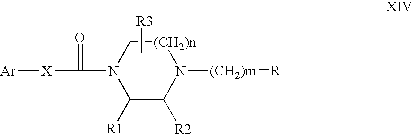
wherein Ar, X, n, m, R₁, R₂, R₃ and R are as defined above,
and reducing them to the Ar-alkyl compounds of formula I and optionally converting them into the physiologically acceptable acid addition salts thereof.

23. A process for the production of compounds of formula I 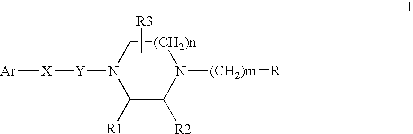
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
the method comprising the steps of alkylating cyclic secondary amines of formula XI wherein n, m, R₁, R₂, R₃ and R are as defined above with formyl derivatives of formula XV
Ar—X—CHO XV
wherein Ar and X are as defined above to compounds of formula I wherein Y is CH₂ and Ar, X, n, m, Ri, R2, R3 and R are as defined above and optionally converting them into the physiologically acceptable acid addition salts thereof.

24. A process for the production of compounds of formula I 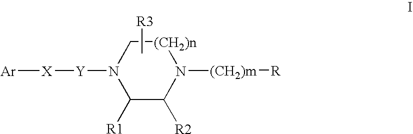
wherein
Ar is naphthyl, a 5-6-membered monocyclic heteroaryl having 1-3 N-atoms, heteroaryl N-oxide, C₄-C₅ heterocycloalkyl having at least 1 N-atom wherein naphthyl, the 5-6-membered monocyclic heteroaryl having 1-3 N-atoms, heteroaryl N-oxide, C₄-C₅ heterocycloalkyl having at least 1 N-atom can be substituted by up to four groups independently selected from hydrogen, fluorine, chlorine, bromine, iodine, trifluoromethyl, halogen substituted C₁₋₆ alkyl, hydroxyl, linear or branched C₁₋₆ alkoxy group, halogen substituted linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
n is an integer from 0 to 3;
m is an integer from 1 to 6, —(CH₂)ₘ— is a linear or branched C₁₋₆ alkyl group;
X is none, —(CH₂)ₚ—, —CHOH—, —CHSH—, CHCN—, —CHOC₁₋₆ alkyl-, —CO—, —SO₂—, —C═C(CN)₂—, wherein p is an integer from 1 to 6, —(CH₂)ₚ— is a linear or branched C₁₋₆ alkyl group;
Y is none, —CH₂—, CO; with the proviso that if n is 1 and X is none, Y is not none;
R₁ and R₂ are both hydrogen or together may form a phenylene ring fused with a piperazine ring (quinoxaline group), a substituted benzene ring fused with a piperazine ring; a heteroaryl ring fused with a piperazine ring, a substituted heteroaryl ring fused with a piperazine ring, a C₄-C₅ heterocycloalkyl ring fused with a piperazine ring; phenylene and heteroaryl can be substituted by up to four groups independently selected from hydrogen, fluorine, chlorine, bromine, iodine, trifluoromethyl, hydroxyl, C₁₋₆ alkoxy group, C₁₋₆ alkyl group, amino, cyano or nitro,
or R₁ and R₂ represent a C₃₋₅ alkylene chain and together with the carbon atoms to which they are attached form a carbocyclic ring;
R₃ is hydrogen, a linear or branched C₁₋₆ alkyl group, fluorine, chlorine, bromine, iodine, trifluoromethyl, halo C₁₋₆ alkyl; and
R is aryl, heteroaryl, cycloalkyl or heterocycloalkyl wherein aryl, heteroaryl, heterocycloalkyl and cycloalkyl can be substituted by up to four groups independently selected from hydrogen, fluorine, chlorine, bromine, iodine, trifluoromethyl, halo C₁₋₆ alkyl, hydroxyl, linear or branched C₁₋₆ alkoxy group, linear or branched C₁₋₆ acyloxy group, phenoxy group, linear or branched C₁₋₆ alkyl group, amino, mono- and di-N—C₁₋₆ alkylamino, acylamino, nitro, cyano, morpholino, carbamoyl, mono- and di-N—C₁₋₆ alkylcarbamoyl, amidino, linear or branched C₁₋₆ alkylamidino, guanidino, linear or branched C₁₋₆ alkylguanidino, ureido, amidoximo, thio, C₁₋₆ alkylthio, C₁₋₆ alkylsulphinyl, C₁₋₆ alkylsulphonyl, carboxyl and C₁₋₆ alkoxycarbonyl group;
the method comprising the steps of coupling cyclic secondary amines of formula XVI wherein Ar, X, Y, n, R₁, R₂ and R₃ are as defined above 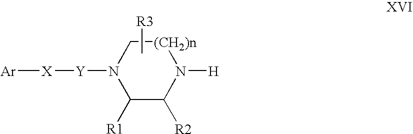
with carboxylic acid derivatives of formula XVII wherein R and m are as defined above,
COOH—(CH₂)m-₁—R XVII
to compounds of formula XVIII wherein Ar, X, Y, n, m, R₁, R₂,R₃ and R are as defined above, 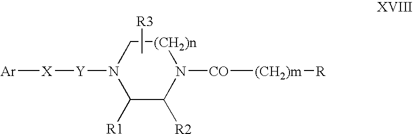
and reducing them to the compounds of formula I and optionally converting them into the physiologically acceptable acid addition salts thereof.
